(19) United States
(12) Patent Application Publication
(10) Pub. No.: US 2013/0321775 A1
(43) Pub. Date: Dec. 5, 2013

(54) DEVICES, SYSTEMS AND METHODS FOR EVALUATION OF THE EYE

(71) Applicant: MUSC FUNDATION FOR RESEARCH DEVELOPMENT, Charleston, SC (US)

(72) Inventors: Elizabeth Richter, Charleston, SC (US); Jan Kylstra, Charleston, SC (US)

(21) Appl. No.: 13/905,988

(22) Filed: May 30, 2013

Related U.S. Application Data
(60) Provisional application No. 61/653,171, filed on May 30, 2012.

(51) Int. Cl. A61B 3/032; A61B 3/12

(52) U.S. Cl. CPC A61B 3/032; A61B 3/12
USPC 351/221; 351/237; 351/246

(57) ABSTRACT

Provided herein are devices, systems and methods for evaluating the eye. For example, the devices, systems and methods are optionally used for evaluating the visual potential of an eye.

### CROSS-REFERENCE TO RELATED APPLICATIONS

This application claims benefit of U.S. Provisional Application No. 61/653,171, filed May 30, 2012, which is hereby incorporated herein by reference in its entirety.

### TECHNICAL FIELD

This document relates to devices, systems and methods for evaluation of the eye. For example, the devices, systems and methods are optionally used for evaluating the visual potential of an eye.

### BACKGROUND

The gold standard for measuring visual acuity is currently the Snellen eye chart at 20 feet. Often opacities or disruptions in the visual axis, such as cataracts or corneal opacities, can decrease visual acuity. In the preoperative assessment of cataracts, practitioners try to determine if the cataract is the sole problem contributing to the diminished visual acuity or if there are concurrent retinal problems that are contributing to the poor eyesight. Since cataracts are usually not homogenous, they frequently have small windows through which images can be projected. The current Marco Guyton-Minkowski potential acuity meter (PAM) determines a patient's potential visual acuity by projecting a small eye chart on the patient's retina. The eye chart is projected on the retina while the PAM is connected to the slit lamp, yet the physician cannot see the projected eye chart and has to rely on the patient's response to determine if the eye chart is being seen. The current PAM device relies largely on patient cooperation that results in the frequent inability to use this method; the cause of failure is not known if it is true retinal pathology or difficulties with equipment use.

### SUMMARY

Provided are systems, devices and methods for evaluating the eye. For example, the devices, system and methods are optionally used for evaluating the visual potential of an eye.

An example device is provided for projecting a visual acuity chart onto the retina of a patient while visualizing the location of the projected chart on the retina. The example device includes a visual acuity chart and a condensing lens aligned with and spaced a distance from the visual acuity chart. The condensing lens is located between the retina of the patient and the visual acuity chart when the device is directed towards the retina for use. The device further includes a viewing area for visualizing an image of the retina in the plane of the visual acuity chart. The example device is referred to herein as the Kylstra-Richter SCALE device, the K-R device, the K-R SCALE, or just “SCALE.” These terms are used interchangeably and are meant to be synonymous.

Optionally, the visual acuity chart comprises one or more letters or symbols. Optionally, the letters or symbols are graduated in size. Optionally, the condensing lens is configured for use in indirect ophthalmoscopy procedures. Optionally, the condensing lens power is about 14 diopters (D) to about 40 diopters (D). For example, the condensing lens power is optionally about 20 D.

The visual acuity chart is optionally located on a surface that allows passage of light through said surface. The surface is in the plane of the image of the retina.

The device optionally further includes a light source suitable for performing indirect ophthalmoscopic visualization of the retina. Optionally, the device comprises a binocular indirect ophthalmoscope.

The visual acuity chart and the condensing lens are aligned such that light passing through the visual acuity chart is projected through the condensing lens and into the subject eye when in use. The light passing through the visual acuity chart causes projection of the visual acuity chart onto the retina of the subject eye.

Optionally, the distance between condensing lens and the visual acuity chart is adjustable. The distance between the condensing lens and the visual acuity chart is optionally varied based on the power of the condensing lens. For example, the distance between the condensing lens and the visual acuity chart is selected such that the image of the retina is produced at the plane of the visual acuity chart. Optionally, the distance between the condensing lens and the visual acuity chart is varied based on the refractive error of at least one eye of the subject.

Also provided is a method for evaluating retinal function in a subject. An example method includes projecting a visual acuity chart from a plane onto the retina and visualizing an image of the retina in the plane of the visual acuity chart. Optionally the subject has a cataract. Optionally, the subject has a corneal lesion. Optionally, the corneal lesion is a scar.

Optionally, evaluating retinal function comprises determining the visual potential of the eye of the subject in which the retina is located.

The visual acuity chart optionally comprises one or more letters or symbols. The letters or symbols are optionally graduated in size. Optionally, the condensing lens is configured for use in indirect ophthalmoscopy procedures. Optionally, the condensing lens power is about 14 diopters (D) to about 40 diopters (D). For example, the condensing lens power is optionally about 20 D.

The visual acuity chart is optionally located on a surface that allows passage of light through said surface. The surface is in the plane of the image of the retina.

The method optionally further includes use of a light source suitable for performing indirect ophthalmoscopic visualization of the retina. Optionally, the method further comprises use of a binocular indirect ophthalmoscope.

The visual acuity chart and the condensing lens are aligned such that light passing through the visual acuity chart is projected through the condensing lens and into the subject eye when in use. The light passing through the visual acuity chart causes projection of the visual acuity chart onto the retina of the subject eye.

Optionally, the distance between condensing lens and the visual acuity chart is adjustable. The distance between the condensing lens and the visual acuity chart is optionally varied based on the power of the condensing lens. For example, the distance between the condensing lens and the visual acuity chart is selected such that the image of the retina is produced at the plane of the visual acuity chart. Optionally, the distance between the condensing lens and the visual acuity chart is varied based on the refractive error of at least one eye of the subject.

Also provided is a system for projecting a visual acuity chart onto the retina of a patient while visualizing the location of the projected chart on the retina. The system includes a light source for performing indirect ophthalmoscopy. The system further includes a visual acuity chart positioned in the pathway of light projected from the light source. The passage of light through the visual acuity chart causes projection of the visual acuity chart onto the retina of the patient. The system further includes a condensing lens aligned with and spaced a distance from the visual acuity chart. The condensing lens is located between the retina of the patient and the visual acuity chart when the device is directed towards the retina for use. While in this location, the lens is positioned in the pathway of light projected from the light source. The system further includes a viewing area for visualizing an image of the retina in the plane of the visual acuity chart.

The visual acuity chart and the condensing lens are aligned such that light passing through the visual acuity chart is projected through the condensing lens and into the subject eye when in use. The light passing through the visual acuity chart causes projection of the visual acuity chart onto the retina of the subject eye.

Optionally, the distance between condensing lens and the visual acuity chart is adjustable. The distance between the condensing lens and the visual acuity chart is optionally varied based on the power of the condensing lens. For example, the distance between the condensing lens and the visual acuity chart is selected such that the image of the retina is produced at the plane of the visual acuity chart. Optionally, the distance between the condensing lens and the visual acuity chart is varied based on the refractive error of at least one eye of the subject.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates an example device for evaluating the eye of a patient.

FIGS. 2A and 2B illustrates the example device of FIG. 1 in use to evaluate the eye of a patient.

FIG. 3 illustrates a physician's view of the example device of FIGS. 1, 2A and 2B.

FIG. 4 shows a comparison of measured visual acuity with standard deviation in logMAR scale.

FIG. 5 shows predicted visual acuity measured by the PAM (squares) and Kylstra Richter SCALE (also referred to herein as K-R SCALE, or simply “SCALE”) (circles) when compared to the actual postoperative visual acuity. The solid line shows what a perfect predictive test would be (x=y). As shown, the K-R SCALE values lie closer to the desired result (lower down on the plot). Regression analyses of the data show a much higher predictive value of the SCALE (r2=0.2043) than the existing gold standard PAM (r2=0.00648). Furthermore, this SCALE regression curve intersects the y axis at 0.2375, which is close to the minimum acuity line on the current version of the SCALE (0.2), showing even better correlation than is able to be measured.

Like reference symbols in the various drawings indicate like elements.

### DETAILED DESCRIPTION

Provided are devices, systems and methods for evaluation of the eye. For example, the devices, systems and methods are optionally used for evaluating the visual potential of an eye. The described devices, systems and methods optionally combine current indirect ophthalmoscopy equipment with a transparent eye chart. A simplistic model of indirect ophthalmoscopy involves the physician wearing a light attached to a headband and holding a 20-diopter lens approximately 3 cm in front of the patient's eye so that a magnified image of the retina can be viewed. An image is formed approximately 5 cm in front of the magnifying lens, and in this same plane an eye chart can be held. This allows the physician to directly project the eye chart onto the patient's retina while the physician also views the patient's retina to make sure the image is directed on the macula. The devices, systems and method are optionally used for determining potential acuity. Indirect ophthalmoscopy is done routinely on clinic patients as part of the retinal exam, so no new risks are introduced to the patient. The devices, systems and methods allow for correctly establishing potential acuity while also allowing the physician to visualize the retina for pathology.

The devices, systems and methods are optionally used by ophthalmologists to better estimate the visual potential of a subject before cataract surgery and thus to improve patient selection for cataract surgery. The methods limit unnecessary cataract surgeries on patients with concomitant retinal issues which limit vision (with or without the cataract) and thus decrease taking unnecessary risks associated with these surgeries. The current Marco Guyton-Minkowski PAM device relies largely on patient cooperation which results in frequent inability to use the method and the cause of failure is not know if it is true retinal or neurologic pathology or difficulties with equipment use. By directly projecting an eyechart onto the patient's retina while the physician simultaneously views the patient's retina to confirm the image is directed on the macula the physician can better distinguish retinal or neurological pathology from equipment failure or lack of cooperation by the patient. The combination of retinal visualization and projection of an eye chart (e.g. visual acuity chart) onto the macula results in a better estimation of potential visual acuity. The devices, systems and methods decrease false negatives.

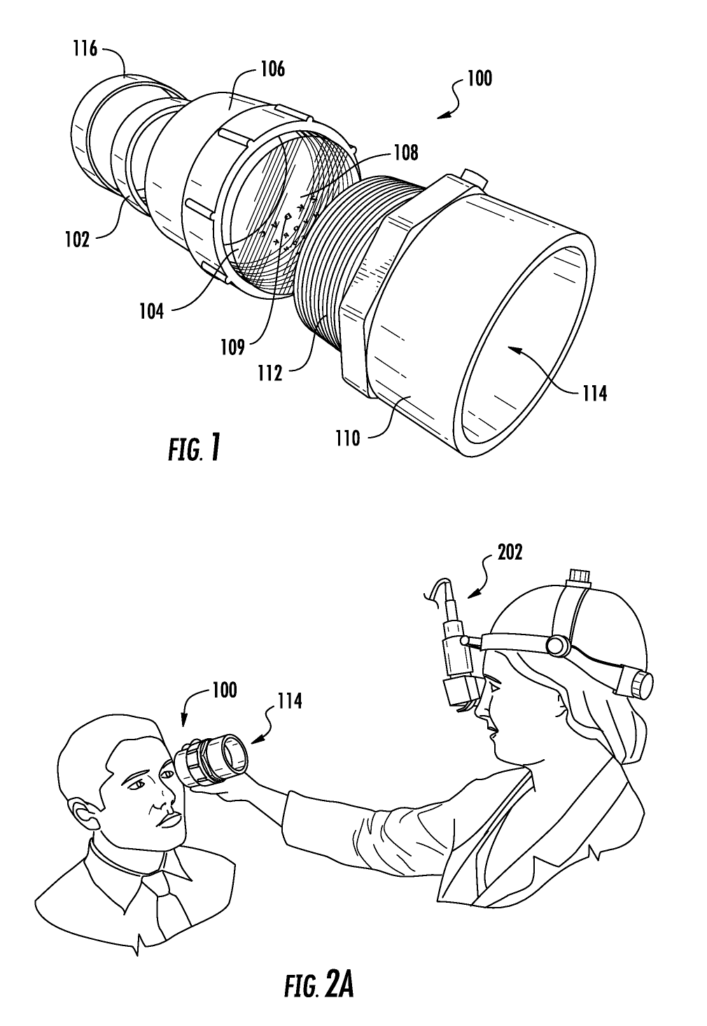

Referring to FIG. 1, an example device 100 is provided for projecting a visual acuity chart 108 onto the retina of a patient while visualizing the location of the projected chart on the retina.

The example device 100 includes a visual acuity chart 108 and a condensing lens 102 aligned with and spaced a distance from the visual acuity chart. The condensing lens 102 is located between the retina of the patient and the visual acuity chart 108 when the device is directed towards the retina for use. The device further includes a viewing area 114 for visualizing an image of the retina in the plane of the visual acuity chart.

Optionally, the visual acuity chart 108 comprises one or more letters or symbols 109. Optionally, the letters or symbols 109 are graduated in size. The visual acuity chart can be switched and different charts are optionally used with the device as desired.

The visual acuity chart is optionally printed on a transparency 104. For example, the print design can optionally be Adobe Indesign CS6, preexisting scan as background layer to ensure size/spacing, Sloan font, and exported to .pdf. The transparency material can be, for example, 0.005 Xpedx Colorlok or 0.007 Mac Papers. A Xerox 242 printer is optionally used with black toner only or dry CMYK toner. The printing can be fused to the printing surface.

Optionally, the condensing lens 102 is configured for use in indirect ophthalmoscopy procedures. Optionally, the condensing lens power is about 14 diopters (D) to about 40 diopters (D). For example, the condensing lens power is optionally about 20 D. The lens 102 can be held in its position at the end of the structure 106 by a friction fit lens guard, such as a round hard plastic insert or a rubber-type O-ring.

The visual acuity chart 108 is optionally located on a surface 104 that allows passage of light through said surface. The surface is in the plane of the image of the retina.

The device 100 optionally further includes a light source suitable for performing indirect ophthalmoscopic visualization of the retina. Optionally, the device comprises a binocular indirect ophthalmoscope 202. Binocular indirect ophthalmoscopy is performed with a binocular indirect ophthalmoscope or BIO. There are several different makes of BIO's available. Although they vary in their position, they have the same basic controls. The eyepieces adjust for the interpupillary distance. There is a lever that changes the spot size of the light, and there is a lever that changes the light color. It varies through white, cobalt blue, and green (no red filter). Some have a yellow filter to decrease retinal damage. Binocular indirect ophthalmoscopy is optionally used with the devices described herein, which include a condensing lens 102. As described above, these lenses typically vary in diopter power from 14-40 D. Binocular indirect ophthalmoscopy is performed with the patient at approximately arm's length. After looking at the patient with the headset the lens 102 is placed in the light beam path in front of the eye. In this regard, the light can be directed though the viewing area or opening 114. Because the visual acuity chart 108 is positioned between the light source of the scope and the lens 102 light passing through the lens first passes through the visual acuity chart 108. This results in projection of the visual acuity chart into the eye and the physician can adjust the location such that it is directed onto the retina.

The lens 102 is optionally moved toward or away from the eye in order to obtain a full view of the fundus. The lens 102 is optionally kept perpendicular to a ray between the observer's eye and the patient's eye. Advantages of binocular indirect ophthalmoscopy include a much wider field of view than can be obtained with direct ophthalmoscopy. This method also provides stereopsis so that lesions can be visualized three-dimensionally. A monocular indirect ophthalmoscope, penlight, or other suitable light sources can be used.

The visual acuity chart 108 and the condensing lens 102 are aligned such that light passing through the visual acuity chart is projected through the condensing lens and into the subject eye when in use. The light passing through the visual acuity chart causes projection of the visual acuity chart, and the physician can direct such projection onto the retina of the subject eye.

Optionally, the distance between condensing lens 102 and the visual acuity chart 108 is adjustable. For example, the visual acuity chart can be located in a tubular structure 110. The tubular structure 110 can be advanced into a second tubular structure 106. Since the lens 102 is located at the end of the tube 106, advancing the tubular structure, containing the visual acuity chart, into 106 causes the visual acuity chart 108 to come closer to the lens 102. Similarly, the structure 110 can be backed out of the structure 106 to separate, or increase the distance between, the visual acuity chart 108 and the lens 102. This way the distance between the lens 102 and the chart 108 can be dynamically adjusted such that the chart is in focus on the retina and the retinal image is created on the plane of the chart.

The distance between the condensing lens and the visual acuity chart is optionally varied based on the power of the condensing lens. For example, the distance between the condensing lens and the visual acuity chart is selected such that the image of the retina is produced at the plane of the visual acuity chart. Optionally, the distance between the condensing lens and the visual acuity chart is varied based on the refractive error of at least one eye of the subject. The device is optionally compact, for example approximately 4×3×3 inches and is light in weight.

Also provided is a method for evaluating retinal function in a subject. An example method includes projecting a visual acuity chart 108 from a plane onto the retina and visualizing an image of the retina in the plane of the visual acuity chart. Optionally, the subject has a cataract or ocular media opacity. Optionally, the subject has a corneal pathology resulting in loss of corneal refractive precision. Optionally, the corneal pathology is a scar, keratoconus, or irregular astigmatism. Optionally the subject has a cataract. Optionally, the subject has a corneal lesion. Optionally, the corneal lesion is a scar. Optionally, the subject reads the visual acuity chart projected onto the retina.

Optionally, evaluating retinal function comprises determining the visual potential of the eye of the subject in which the retina is located.

The visual acuity chart 108 optionally comprises one or more letters or symbols. The letters or symbols are optionally graduated in size. Optionally, the method includes use of a condensing lens 102 that is configured for use in indirect ophthalmoscopy procedures. Optionally, the condensing lens power is about 14 diopters (D) to about 40 diopters (D). For example, the condensing lens power is optionally about 20 D.

The visual acuity chart 108 is optionally located on a surface that allows passage of light through said surface. The surface is in the plane of the image of the retina.

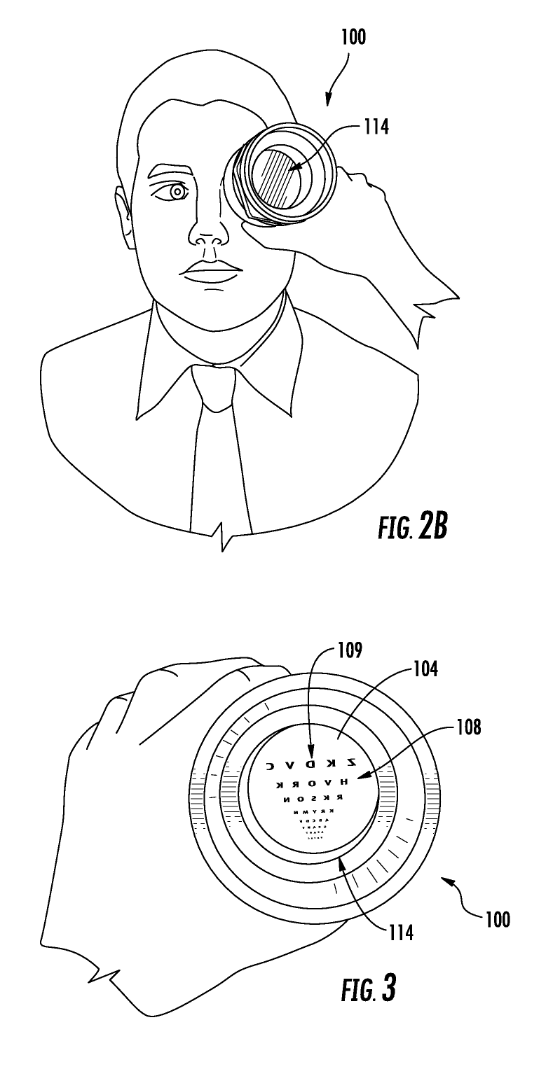

Referring to FIGS. 2A and 2B, the method optionally further includes use of a light source 202 suitable for performing indirect ophthalmoscopic visualization of the retina. Optionally, the method further comprises use of a binocular indirect ophthalmoscope.

The visual acuity chart 108 and the condensing lens are aligned such that light passing through the visual acuity chart is projected through the condensing lens 102 and into the subject eye when in use. The light passing through the visual acuity chart 108 causes projection of the visual acuity chart onto the retina of the subject eye. The physician can view an image of the retina as with indirect ophthalmoscopy while confirming and visualizing the chart on the retina. Thus, by directly projecting an eyechart onto the patient's retina while the physician simultaneously views the patient's retina to confirm the image is directed on the macula the physician can better distinguish retinal or neurological pathology from equipment failure or lack of cooperation by the patient. The combination of retinal visualization and projection of an eye chart (e.g. visual acuity chart) onto the macula results in a better estimation of potential visual acuity. The devices, systems and methods decrease false negatives.

Optionally, the distance between condensing lens and the visual acuity chart is adjustable. The distance between the condensing lens and the visual acuity chart is optionally varied based on the power of the condensing lens. For example, the distance between the condensing lens and the visual acuity chart is selected such that the image of the retina is produced at the plane of the visual acuity chart. Optionally, the distance between the condensing lens and the visual acuity chart is varied based on the refractive error of at least one eye of the subject.

Referring to FIGS. 2A and 2B, also provided is a system for projecting a visual acuity chart 108 onto the retina of a patient while visualizing the location of the projected chart on the retina. The system includes a light source 202 for performing indirect ophthalmoscopy. The system further includes a visual acuity chart 108 positioned in the pathway of light projected from the light source. The passage of light through the visual acuity chart causes projection of the visual acuity chart onto the retina of the patient. The system further includes a condensing lens 102 aligned with and spaced a distance from the visual acuity chart 108. The condensing lens is located between the retina of the patient and the visual acuity chart when the device is directed towards the retina for use. While in this location, the lens is positioned in the pathway of light projected from the light source 202. The system further includes a viewing area 114 for visualizing an image of the retina in the plane of the visual acuity chart 108.

The visual acuity chart 108 and the condensing lens 102 are aligned such that light passing through the visual acuity chart is projected through the condensing lens and into the subject eye when in use. The light passing through the visual acuity chart causes projection of the visual acuity chart onto the retina of the subject eye.

Optionally, the distance between condensing lens 102 and the visual acuity chart 108 is adjustable. The distance between the condensing lens 102 and the visual acuity chart 108 is optionally varied based on the power of the condensing lens. For example, the distance between the condensing lens and the visual acuity chart is selected such that the image of the retina is produced at the plane of the visual acuity chart. Optionally, the distance between the condensing lens and the visual acuity chart is varied based on the refractive error of at least one eye of the subject.

### EXAMPLES

### Example 1

Twelve normal undilated eyes were evaluated using the above described devices, systems and methods. Ten of twelve could read the lowest line of the visual acuity chart (20/32 vision). The remaining two were able to read the next line.

### Example 2

Twenty five patients with visually significant cataracts were evaluated using the above described devices, systems and methods, referred to in this example as the K-R SCALE. An acuity comparison was made between the Marco Guyton-Minkowski PAM, post surgical Snellen acuity, Pinhole and the described devices, systems and methods. Patient 1 was a 65 year old white male with brunescent cataracts OU. Table 1 shows results for this patient.

TABLE 1

| | OD | OS |
|---|---|---|
| Snellen | CF@3′ | CF@3′ |
| PAM | unable | 20/300 |
| K-R SCALE | 20/100 | 20/30 |

Patient 2 was a 63 year old white female with 3+CORT, 3+PSC OS. Table 2 shows results for this patient.

TABLE 2

| | OD | OS |
|---|---|---|
| Snellen | | CF@6′ |
| PAM | | 20/100, 20/30 dilated |
| 20/30-dilated | | 20/30 |

### Example 3

Visual acuity measurement of the Kylstra-Richter SCALE was determined when compared to Guyton-Minkowski PAM in 23 patients with preoperative cataracts. The overall goal of this clinical trial was to determine ease of use and correlation with other accepted methods.

Informed consent was obtained on each of 23 patients (mean age 69 years, range 55-84 years) preparing to undergo cataract surgery. On each patient, measurement of best corrected visual acuity (BCVA) using a Snellen wall chart at 20 feet was collected, as well as an undilated Guyton-Minkowski PAM acuity (detectable range 20/20 to 20/800) and a dilated Kylstra-Richter SCALE (also referred to herein as K-R SCALE, or simply “SCALE”) acuity (detectable range 20/32 to 20/252) Post-surgical data was also evaluated. The results are shown in Table 3. After conversion of visual acuity to logMAR, paired t-test was used to compare differences amongst the data. The results are shown in Table 4.

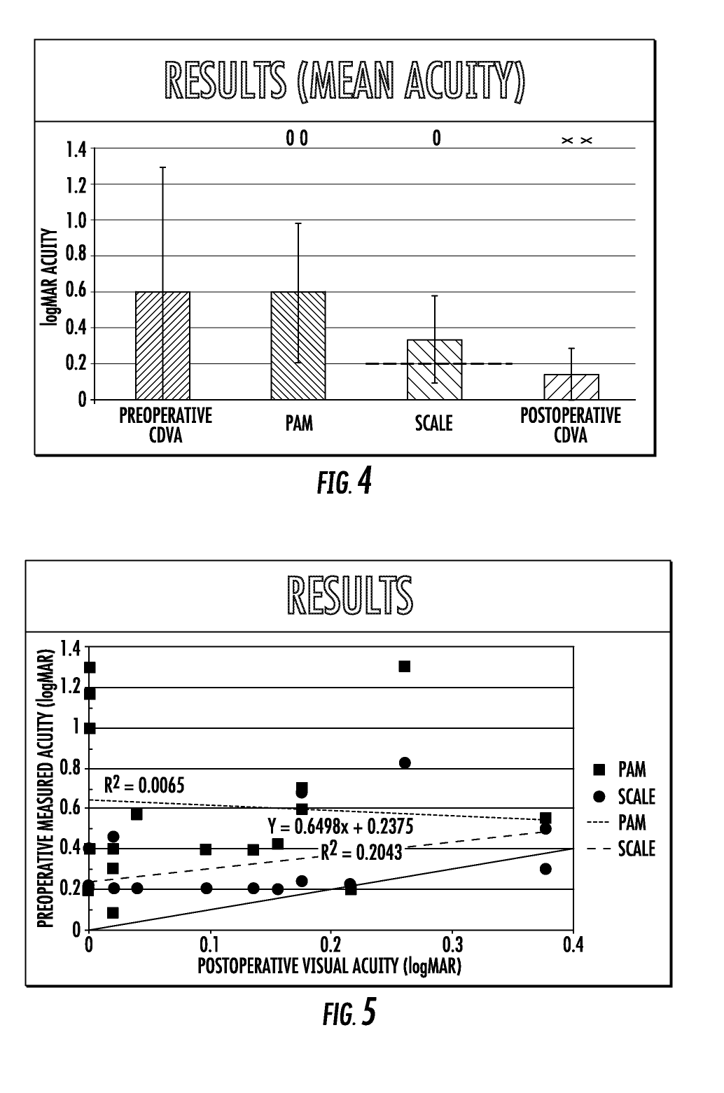

Of 23 eyes, only one patient (ID#23) was unable to see letters on either device. Another subject (ID#14) was unable to see anything on the PAM device (>20/800), but could see one line (20/252-1) with the SCALE (also referred to herein as K-R SCALE, or Kylstra-Richter SCALE). Analysis of the other patients yielded a significant difference between the visual acuities measured by these two acuity methods (t(20)=3.4916, p=0.00230). The mean PAM logMAR acuity was 0.589 (SD=0.389),while the yielded a 0.335 mean acuity (SD=0.246); this is approximately 2-line increase on the ETDRS chart. Results can be seen in FIGS. 4 and 5.

TABLE 3

| | | | Measured acuity | | | |
|---|---|---|---|---|---|---|
| | | | Snellen | | | Post-surgery |
| | | | BCVA | | | Snellen |
| ID | Age | Eye | (20′) | PAM | SCALE | (20′) |
| 1 | 73 | OD | 20/70 − 2 | 20/70 − 1 | 20/32 | 20/20 − 2 |
| 2 | 74 | OD | 20/20 | 20/30 − 1 | 20-32 | 20/20 |
| 3 | 65 | OD | 20/60 | 20/50 | 20/32 | 20/30 + 2 |
| 4 | 66 | OS | 20/60 | 20/40 | 20/63 + 2 | 20/20 − 1 |
| 5 | 73 | OS | 20/25 | 20/30 − 1 | 20/32 − 1 | 20/30 − 2 |
| 6 | 77 | OS | 20/50 | 20/400 | 20/32 − 1 | 20/20 |
| 7 | 70 | OD | 20/200 | 20/400 | 20/126 − 1 | 20/40 − 2 |
| 8 | 55 | OS | 20/60 | 20/80 + 1 | 20/32 − 2 | 20/30 |
| 9 | 69 | OS | 20/25 | 20/300 | 20/32 − 1 | 20/20 |
| 10 | 64 | OD | 20/25 − 2 | 20/50 | 20/32 | 20/25 |
| 11 | 84 | OS | 20/40 | 20/50 | 20/50 | 20/20 |
| 12 | 64 | OS | 20/50 | 20/200 | 20/32 | 20/20 |
| 13 | 67 | OS | 20/30 | 20/100 | 20/100 + 1 | 20/30 |
| 14 | 75 | OD | CF at 3′ | unable | 20/252 − 1 | 20/50 |
| 15 | 77 | OS | 20/40 | 20/50 − 1 | 20/32 | 20/30 + 1 |
| 16 | 68 | OS | 20/25 | 20/25 + 1 | 20/32 | 20/20 − 1 |
| 17 | 64 | OD | 20/50 | 20/50 | 20/32 | 20/20 − 1 |
| 18 | 76 | OD | 20/40 | 20/70 | 20/63 | 20/50 + 1 |
| 19 | 61 | OD | 20/25 + 3 | 20/20 − 2 | 20/32 | ns |
| 20 | 61 | OD | 20/40 | 20/60 − 1 | 20/40 | 20/50 − 1 |
| 21 | 65 | OS | CF at 3′ | 20/300 | 20/32 | ns |
| 22 | 63 | OS | CF at 6′ | 20/100 | 20/32 | ns |
| 23 | 61 | OS | HM | unable | unable | 20/40 − 1 |

TABLE 4

| | | | logMAR | | | |
|---|---|---|---|---|---|---|
| ID | Age | Eye | BCVA | PAM | SCALE | Post-surgery |
| 1 | 73 | OD | 0.504 | 0.564 | 0.2 | 0.04 |
| 2 | 74 | OD | 0.000 | 0.196 | 0.2 | 0 |
| 3 | 65 | OD | 0.477 | 0.398 | 0.2 | 0.136 |
| 4 | 66 | OS | 0.477 | 0.301 | 0.46 | 0.02 |
| 5 | 73 | OS | 0.097 | 0.196 | 0.22 | 0.216 |
| 6 | 77 | OS | 0.398 | 1.301 | 0.22 | 0 |
| 7 | 70 | OD | 1.000 | 1.301 | 0.82 | 0.261 |
| 8 | 55 | OS | 0.477 | 0.582 | 0.24 | 0.176 |
| 9 | 69 | OS | 0.097 | 1.176 | 0.22 | 0 |
| 10 | 64 | OD | 0.057 | 0.398 | 0.2 | 0.097 |
| 11 | 84 | OS | 0.301 | 0.398 | 0.4 | 0 |
| 12 | 64 | OS | 0.398 | 1 | 0.2 | 0 |
| 13 | 67 | OS | 0.176 | 0.699 | 0.68 | 0.176 |
| 14 | 75 | OD | 1.900 | unable | 1.12 | 0.398 |
| 15 | 77 | OS | 0.301 | 0.418 | 0.2 | 0.156 |
| 16 | 68 | OS | 0.097 | 0.077 | 0.2 | 0.02 |
| 17 | 64 | OD | 0.398 | 0.398 | 0.2 | 0.02 |
| 18 | 76 | OD | 0.301 | 0.544 | 0.5 | 0.378 |
| 19 | 61 | OD | 0.157 | 0.04 | 0.2 | ns |
| 20 | 61 | OD | 0.301 | 0.497 | 0.3 | 0.378 |
| 21 | 65 | OS | 1.900 | 1.176 | 0.2 | ns |
| 22 | 63 | OS | 1.800 | 0.7 | 0.2 | ns |
| 23 | 61 | OS | 2.300 | unable | unable | 0.321 |

A number of embodiments of the invention have been described. Nevertheless, it will be understood that various modifications may be made without departing from the spirit and scope of the invention. Accordingly, other embodiments are within the scope of the following claims.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a method is disclosed and discussed each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A device for projecting a visual acuity chart onto the retina of a patient while visualizing the location of the projected chart on the retina, comprising:
a. a visual acuity chart;
b. a condensing lens aligned with and spaced a distance from the visual acuity chart, wherein the condensing lens is located between the retina of the patient and the visual acuity chart when the device is directed towards the retina for use; and
c. a viewing area for visualizing an image of the retina in the plane of the visual acuity chart.

2. The device of claim 1, wherein the visual acuity chart comprises one or more letters or symbols.

3. The device of claim 2, wherein the letters or symbols are graduated in size.

4. The device of claim 1, wherein the condensing lens is configured for use in indirect ophthalmoscopy procedures.

5. The device of claim 4, wherein the condensing lens power is about 14 diopters (D) to about 40 diopters (D).

6. The device of claim 1, wherein the visual acuity chart is located on a surface that allows passage of light through said surface.

7. The device of claim 7, wherein the surface is in the plane of the image of the retina.

8. The device of claim 1, further comprising a light source suitable for performing indirect ophthalmoscopic visualization of the retina.

9. The device of claim 1, further comprising a binocular indirect ophthalmoscope, a monocular indirect ophthalmoscope, or a penlight.

10. The device of claim 1, wherein the visual acuity chart and the condensing lens are aligned such that light passing through the visual acuity chart is projected through the condensing lens and into the subject eye when in use.

11. The device of claim 10, wherein light passing through the visual acuity chart causes projection of the visual acuity chart onto the retina of the subject eye.

12. The device of claim 1, wherein the distance between condensing lens and the visual acuity chart is adjustable.

13. The device of claim 1, wherein the distance between the condensing lens and the visual acuity chart is varied based on the refractive error of at least one eye of the subject.

14. The device of claim 1, wherein the distance between the condensing lens and the visual acuity chart is varied based on the power of the condensing lens.

15. The device of claim 14, wherein the distance between the condensing lens and the visual acuity chart is selected such that the image of the retina is produced at the plane of the visual acuity chart.

16. A method for evaluating retinal function in a subject, comprising:
a. projecting a visual acuity chart onto the retina; and
b. visualizing an image of the retina in the plane of the visual acuity chart.

17. The method of claim 16, wherein the subject has a cataract or ocular media opacity.

18. The method of claim 16, wherein evaluating retinal function comprises determining the visual potential of the eye of the subject in which the retina is located.

19. The method of claim 16, further comprising positioning a condensing lens configured for use in indirect ophthalmoscopy procedures between the visual acuity chart and the retina.

20. A system for projecting a visual acuity chart onto the retina of a patient while visualizing the location of the projected chart on the retina, comprising:
a. a light source for performing indirect ophthalmoscopy;
b. a visual acuity chart positioned in the pathway of light projected from the light source, wherein the passage of light through the visual acuity chart causes projection of the visual acuity chart onto the retina of the patient;
c. a condensing lens aligned with and spaced a distance from the visual acuity chart, wherein the condensing lens is located between the retina of the patient and the visual acuity chart when the device is directed towards the retina for use, wherein the lens is positioned in the pathway of light projected from the light source; and
d. a viewing area for visualizing an image of the retina in the plane of the visual acuity chart.
